(19) United States
(12) Patent Application Publication
(10) Pub. No.: US 2004/0013669 A1
(43) Pub. Date: Jan. 22, 2004

(54) Method for treating persistent pain and identifying compounds to treat persistent pain

(76) Inventors: Feng Wei, Ellicott City, MD (US); Daniel R. Storm, Seattle, WA (US); Louis J. Muglia, St. Louis, MO (US); Min Zhuo, St. Louis, MO (US)

Correspondence Address: Michael T. Marrah, Sonnenschein Nath & Rosethal LLP, Wacker Drive Station, Sears Tower, P.O. Box #061080, Chicago, IL, 60606-1080, US

(21) Appl. No.: 10/460,065

(22) Filed: Jun. 11, 2003

Related U.S. Application Data
(60) Provisional application No. 60/388,395, filed on Jun. 12, 2002.

(51) Int. Cl. A61K 39/395

(52) U.S. Cl. 424/146.1

(57) ABSTRACT

Disclosed is method of down-regulating an activity associated with AC1, AC8 or both in a subject comprising administering to the subject an antagonist to AC1, AC8 or both in an amount sufficient to affect the antagonism.

### CROSS REFERENCE TO RELATED APPLICATION

This invention claims the benefit, under Title 34, United States Code 119(e) of Provisional Application No. 60/388,395, filed Jun. 12, 2002 entitled “Method for Treating Persistent Pain” which is hereby incorporated by this reference.

### BACKGROUND OF THE INVENTION

Throughout this application various publications are referenced. The disclosures of these publications in their entireties are hereby incorporated by reference in this application in order to more fully describe the sate of the art to which this invention pertains.

1. Field of the Invention

This invention relates to the field of medicine, biochemistry, and neuroscience. In particular, the invention provides methods for treating persistent pain, such as inflammation-related allodynia.

2. Discussion of the Related Art

Tissue injury or damage often causes persistent pain in patients. Generally pain is experienced when the free nerve endings which constitute the pain receptors in the skin as well as in certain internal tissues are subjected to mechanical, thermal or chemical stimuli. The pain receptors transmit signals along afferent neurons into the spinal cord and thence to the brain.

The causes of pain can include inflammation, injury, disease, muscle spasm and the onset of a neuropathic event or syndrome. Ineffectively treated pain can be devastating to the person experiencing it by limiting function, reducing mobility, complicating sleep, and dramatically interfering with the quality of life.

Inflammatory pain can occur when tissue is damaged, as can result from surgery or due to an adverse physical, chemical or thermal event or to infection by a biologic agent. Neuropathic pain is a persistent pain syndrome that can result from damage to the nervous system, the peripheral nerves, the dorsal root ganglion or dorsal root, or to the central nervous system. It includes pain caused by an increased sensitivity to noxious stimuli (called hyperalgesia) and pain caused by previously innocuous stimuli (called allodynia). Hyperalgesia and allodynia often spread into neighboring or remote parts of body due to long-term changes in the central nervous system (Woolf, 1983; Woolf and Salter, 2000). In severe cases of allodynia, even wearing clothes can become extremely painful. While peripheral and central mechanisms are thought to play important roles, specific signaling molecules remain to be identified.

Coincidence detection and crosstalk between signal transduction systems are thought to be important for physiological functions of the brains (Xi and Strom, 1997). cAMP signal pathways are important for many brain functions such as learning and memory, drug addiction and pain (Kandel and Schawatz, 1982; Nestler and Aghajanian, 1997; Malmberg et al., 1997; Woolf and Salter, 2000). Although many molecules have been reported to be involved in injury-related hyperalgesia and pain induced by non-noxious stimuli, there has been no report of the deletion or inhibition of a signal molecule to eliminate allodynia or other persistent pain.

Calmodulin (“CaM”)-regulated adenylyl cyclases (“ACs”) serve as coincidence detectors that couple the Ca²⁺ and cAMP signaling pathways. (Xi and Strom, 1997). AC1 and AC8 are the two CaM stimulated ACs found in the brain (Wong et al., 1999; Xi and Strom, 1997). In both hippocampus and cerebellum, AC1 or AC8 KO significantly reduced Ca²⁺-stimulated ACs and no measurable Ca²⁺-stimulated AC activity was found in AC 1&8 DKO mice (see Wong et al., 1999). AC1 and AC8 have different sensitivities to Ca²⁺ (Xi and Strom, 1997). AC1 is four to five times more sensitive to Ca²⁺ than AC8 (Cali et al., 1996).

There remains a definite need to identify signal molecules responsible for the induction and expression of persistent pain. There remains a further definite need for a method for targeting such signal molecules to treat unwanted persistent pain, such as allodynia in humans.

### SUMMARY OF THE INVENTION

Now in accordance with the invention, there has been found a method of down-regulating an activity associated with AC1, AC8 or both in a subject. In some embodiments, the activity is an activity in the forebrain of the subject. The inventive method is useful in inhibiting persistent pain, such as persistent pain caused by inflammation related allodynia.

In some embodiments, an antagonist to AC1, AC8 or both is administered to the subject in an amount sufficient to affect the antagonism. For example, in some embodiments, a therapeutically effective dose of an antagonist to AC1, AC8 or both is administered to a patient suffering from persistent pain in an amount sufficient to inhibit the persistent pain in the patient.

In other embodiments, activity associated with AC1, AC8 or both in a subject is down-regulated by reducing or eliminating expression of AC1, AC8 or both at the transcription level, the translational level or both levels. For example, in some embodiments the activity is down-regulated using a promoter derived from the αCaMKII gene.

In still other embodiments, activity associated with AC1, AC8 or both in a subject is down-regulated by the selective use of a compound that acts inside of the subject's cells, such as cells of the subject's forebrain. In some embodiments, the compound is comprised of somatic cells transformed with a vector encoding an antisense molecule or ribosome, or a transcription suppressing protein designed to inhibit expression of AC1, AC8 or both.

Further in accordance with the invention, there has been found a method of identifying compounds that inhibit persistent pain by down-regulating AC1, AC8 or both. The inventive method includes the steps of contacting a chimeric DNA construct having an AC1, AC8 or both promoter operably linked to a reporter gene with a test compound suspected of down-regulating AC1, AC8 or both and then measuring expression of the reporter gene. A decrease in the expression of the reporter gene in the presence of the compound is indicative that the compound inhibits persistent pain.

Still further in accordance with the invention, there has been found a genetically altered non-human animal having increased sensitivity to persistent pain as compared with an equivalent, but unaltered animal, such that the animal expresses a gene encoding AC1, AC8 or both to a greater extent in the forebrain than does the equivalent, but unaltered animal. In some embodiments, the genetically altered animal overexposes an endogenous gene encoding AC1, AC8 or both.

In other embodiments, the genetically altered animal expresses a transgene encoding AC1, AC8 or both. In some of these embodiments, the genetically altered animal has a transgene that includes the entire coding region of an AC1, AC8 or both gene, or its complementary DNA or chimeric genes containing part or all of an AC1 and/or AC8 coding region. And in some of these embodiments, the transgene includes a promoter derived from the αCaMKII gene.

Still further in accordance with the invention, there has been found an in vivo assay for identifying compounds that inhibit persistent pain by down-regulating AC1, AC8 or both activity. The assay includes the steps of administering to a non-human transgenic animal that expresses a gene encoding AC1, AC8 or both to a greater extent in its forebrain than does the equivalent, but unaltered animal a test compound suspected of down-regulating AC1, AC8 or both and then directly or indirectly measuring an activity associated with AC1, AC8 or both of the treated animal as compared with an equivalent untreated animal, a decrease in the activity of the treated animal being indicative that the test compound reduces or eliminates persistent pain. In some embodiment, the activity is measured using behavioral tests of long term response to a persistent pain stimulus.

Other features and advantages of the present invention will be understood by reference to the figures, detailed description, and examples that follow.

### BRIEF DESCRIPTION OF THE FIGURES

FIG. 1 depicts AC1 and AC8 expression in the ACC, insular cortex, hippocampus and spinal dorsal horn of mice.

FIG. 2 illustrates Ca²⁺-stimulated adenylyl cyclases activity in the spinal cord of double knockout mice.

FIG. 3 illustrates allodynia and reduced nociceptive responses in AC1 and AC8 double knockout mice.

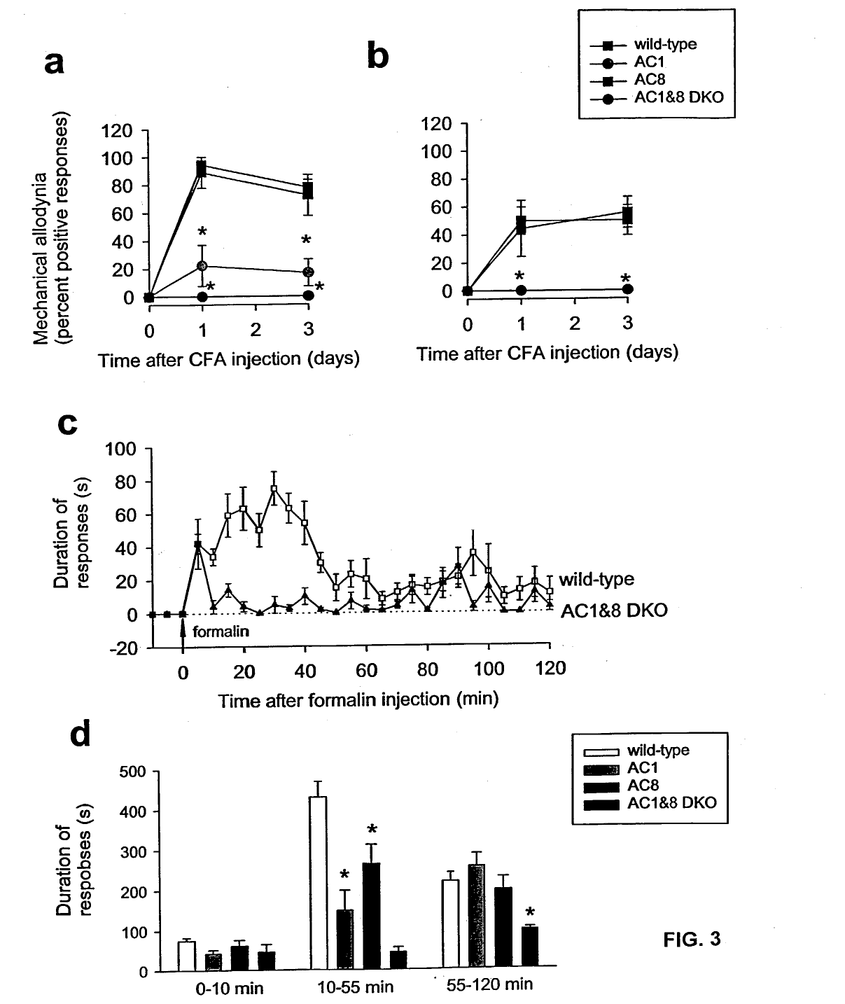

FIG. 4 illustrates the effect of AC1 and AC8 on CREB activation following formalin injection.

### DETAILED DESCRIPTION OF THE PREFERRED EMBODIMENTS

I. Definitions:

Various terms relating to the present invention are used hereinabove and also throughout the specifications and claims.

The terms “coding sequence” or “coding region” refer to a nucleic acid molecule having sequence information necessary to produce a gene product, when the sequence or region is expressed.

The terms “operably linked” or “operably inserted” mean that the regulatory sequences necessary for expression of the coding sequence are placed in a nucleic acid molecule in the appropriate positions relative to the coding sequence so as to enable expression of the coding sequence. This same definition is also applied to the arrangement of other transcription control elements (e.g., enhancers) in an expression vector.

The terms “transcriptional control sequences” and “translational control sequences” refer to DNA regulatory sequences, such as promoters, enhancers, polyadenylation signals, terminators, and the like, that provide for the expression of a coding sequence in a host cell.

The terms “promoter region” or “promoter sequence” refer to transcriptional regulatory regions of a gene, which may be found at the 5′ or 3′ side of the coding region, or within the coding region, or within introns. Typically, a 3′ promoter region or sequence is a DNA regulatory region capable of binding RNA polymerase in a cell and initiating transcription of a downstream (3′ direction) of a coding sequence. The typical 5′ promoter region or sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “vector” refers to a replicon, such as plasmid, phage, cosmid, or virus to which another nucleic acid segment may be operably inserted so as to bring about the replication or expression of the segment.

The terms “nucleic acid construct” or “DNA construct” refer to a coding sequence or sequences operably linked to appropriate regulatory sequences and inserted into a vector for transforming a cell. These terms may be used interchangeably with the terms “transforming DNA” or “transgene”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

The term “heterologous region of a nucleic acid” refers to a construct of an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a coding sequence is a construct where the coding sequence itself is not found in nature (e.g., a cDNA where the genomic coding sequence contains introns or synthetic sequences having codons different than the native gene). Allelic variations or naturally-occurring mutational events do not give rise to a heterologous region of DNA as defined herein.

A cell has been “transformed” or “transfected” by exogenous or heterologous DNA when such DNA has been introduced inside the cell. The transforming DNA (transgene) may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations. If germline cells are stably transformed, the transformation may be passed from one generation of animals arising from the germline cells, to the next generation. In this instance, the transgene is referred to as being inheritable.

The term “subject” as used herein refers to a human subject or a non-human animal subject, or it may refer to any other living organism. The term “patient” may be used interchangeably for the term “subject”.

The term “acute pain” refers to an unpleasant sensation induced by noxious stimuli. It is short-lasting and can occur without any tissue injury.

The term “persistent pain” refers long-lasting, unpleasant sensations that are often related to tissue injury. Persistent pain lasts long after the initial noxious stimulus producing acute pain is gone, and may persist from days to years.

Other definitions are found in the description set forth below.

II. Description:

The present invention encompasses a method of antagonizing AC1 and /or AC8 associated activity in a subject comprising administering to said subject an antagonist to AC1 and/or AC8 in an amount sufficient to affect said antagonism. Also encompassed in the present invention is a method of treating persistent pain in a subject with an antagonist of AC1 and/or AC8-related activity. AC1 and AC8 are essential for central synapses to process sensory information in pathological conditions such as tissue injury and inflammation. Deletion of AC1 and AC8 selectively blocks the induction and expression of chronic pain, such as inflammation-related allodynia.

AC1 and/or AC8-related activities, particularly AC1 and/or AC8-related activities in the forebrain, can be down-regulated by any satisfactory method. Representative methods for inhibiting AC1 and/or AC8-related activities include, but are not limited to: (1) administration of an effective amount of compounds that act directly or indirectly to reduce AC1 and/or AC8-related activities; (2) reducing or eliminating expression of AC1 and/or AC8 at the transcriptional (e.g., promoter) and/or translational level; and (3) use of AC1 or AC8 compounds that act inside cells, particularly cells of the forebrain, to interfere with the interaction between AC1 and/or AC8 and their downstream targets.

In a preferred embodiment, an AC1 and/or AC8 antagonist compound is administered to a subject in an amount sufficient to antagonize AC1 and/or AC8 associated function(s). The particular compound can be administered to a patient either by itself or in a pharmaceutical composition where it is mixed with suitable carriers or excipient(s). In treating a patient, a therapeutically effective dose of the compound is administered. A therapeutically effective dose refers to that amount of the compound that results in the reduction or elimination of persistent pain.

Toxicity and therapeutic efficacy of such compounds can be determined by standard pharmaceutical procedures in cell cultures or experimental animals. Cell culture assays and animal studies can be used for determining the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Compounds which exhibit large therapeutic indices are preferred. The data obtained from these cell culture assays and animal studies can be used in formulating a range of dosages for use in human patients. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays by determining an IC₅₀ (i.e., the concentration of the test compound which achieves a half-maximal inhibition at the cellular level). A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact formulation, route of administration and dosage can be chosen by the individual physician in view of the patient's condition. (See, e.g. Fingl et al., 1975, in “The Pharmacological Basis of Therapeutics”, Ch. 1, p. 1).

The attending physician will know how to and when to terminate, interrupt, or adjust administration due to toxicity, or to organ dysfunctions. Conversely, the attending physician will also know to adjust treatment to higher levels if the clinical response were not adequate (precluding toxicity). The magnitude of an administrated dose will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

The antagonist may be formulated and administered systemically or locally. Techniques for formulation and administration may be found in “Remington's Pharmaceutical Sciences,” 1990, 18th ed., Mack Publishing Co., Easton, Pa. Suitable routes may include oral, or parenteral delivery, including intramuscular, subcutaneous, intramedullary injections, as well as intrathecal, direct intraventricular or intravenous injections, just to name a few.

Use of pharmaceutically acceptable carriers to formulate the antagonists into dosages suitable for systemic administration is within the scope of the invention. With proper choice of carrier and suitable manufacturing practice, the compositions of the present invention, in particular those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Antagonists intended to be administered intracellularly may be administered using techniques well known to those of ordinary skill in the art. For example, such agents may be encapsulated into liposomes, and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Small organic molecules may be directly administered intracellularly due to their hydrophobicity.

Pharmaceutical compositions suitable for use in the present invention include compositions wherein the active ingredients are contained in an amount effective to achieve its intended purpose. Determination of an effective amount is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose,sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Alternatively, somatic cells of subjects may be stably or transiently transformed with a vector encoding an antisense molecule or ribozyme, or a transcription suppressing protein, for instance, designed to inhibit expression of AC1 and/or AC8. Such DNA therapy, for instance, comprises targeted administration of an AC1 and/or AC8 expression-inhibiting vector which, upon delivery to the target cells, inhibits the production of AC1 and/or AC8. In preferred embodiments, DNA therapy to transiently produce such AC1 and/or AC8 expression-inhibiting molecules is targeted to forebrain locations and can be accomplished according to methods well known in the art. For instance, the forebrain may be selectively targeted by using a promoter that is specific for gene expression in that region, such as a promoter derived from the αCaMKII gene, whose activity has been demonstrated to be restricted to the forebrain region (Mayford et al., Cell 81, 891-904, 1995).

The present invention further provides an in vitro assay for identifying compounds that inhibit persistent pain. The compounds inhibit the function of AC1 and/or AC8 in a subject by decreasing expression of AC1 and/or AC8. This assay involves: (1) providing a chimeric DNA construct comprising an AC1 and/or AC8 promoter operably linked to a reporter gene; (2) contacting the chimeric DNA construct with a test compound suspected of down-regulating the AC1 and/or AC8 promoter, and (3) measuring expression of the reporter gene. A decrease in the expression of the reporter gene in the presence of the test compound indicates that the test compound will be useful in the management of persistent pain by decreasing the expression of AC1 and/or AC8 genes.

The present invention still further provides an in vivo system for identifying compounds that inhibit persistent pain by decreasing expression of AC1 and/or AC8 genes. The system includes genetically altered animals having increased sensitivity to persistent pain as compared to equivalent, but unaltered animals, because the animals express a gene, either an endogenous or a transgene, encoding AC1 and/or AC8 to a greater extent in its forebrain than does the equivalent, but unaltered animal.

The term “animal” is used herein to include all vertebrate animals, except humans. It also includes an individual animal in all stages of development, including embryonic and fetal stages. Examples of animals preferred for use in the present invention include, but are not limited to, rodents, most preferably mice and rats, as well as cats, dogs, dolphins and primates, other than humans.

A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule, i.e., a “transgene”.

The term “transgene”, as used herein, refers to any exogenous gene sequence which is introduced into both the somatic and germ cells or only some of the somatic cells of a mammal. This DNA molecule may be specifically targeted to defined genetic locus, or be randomly integrated within a chromosome, or it may be extrachromosomally replicating DNA.

The term “germline transgenic animal” refers to a transgenic animal in which the transgene was introduced into a germline cell, thereby making the genetic alteration inheritable. If such offspring, in fact, possess the transgene then they, too, are transgenic animals.

The transgene of the present invention includes without limitation, the entire coding region of an AC1 and/or AC8 gene, or its complementary DNA (cDNA), or chimeric genes containing part or all of an AC1 and/or AC8 coding region, whose expression in the forebrain is driven by a tissue specific promoter. It is preferable, but not essential, that the AC1 and/or AC8 coding sequence used in the transgene be of the same species origin as the transgenic animal to be created.

Methods to obtain transgenic, non-human mammals are known in the art (e.g., Joyner, “Gene Targeting,” IRL Press, Oxford, 1993; Hogan, et al. (eds.), “Manipulating the Mouse Embryo—A Laboratory Manual,” Cold Spring Harbor Laboratory Press, Cold Spring Harbor, N.Y., 1994; and Wasserman & DePamphilis, “A Guide to Techniques in Mouse Development,” Academic Press, San Diego Calif. 1993.

One method for introducing exogenous DNA into the germline is by microinjection of the gene construct into the pronucleus of an early stage embryo (e.g., before the four-cell stage) (Wagner et al., Proc. Natl. Acad. Sci. USA 78: 5016, 1981; Brinster et al., Proc. Natl. Acad. Sci. USA 82, 4438, 1985). The detailed procedure to produce AC1 and/or AC8 transgenic mice by this method has been described (Tsien et al., Cell, 87: 1317-26. 1996).

Another method for producing germline transgenic mammals utilizes embryonic stem cells. The DNA construct may be introduced into embryonic stem cells by homologous recombination (Thomas et al., Cell 51: 503, 1987; Capecchi, Science 244: 1288, 1989; Joyner, et al., Nature 338: 153, 1989) in a transcriptionally active region of the genome. A suitable construct may also be introduced into the embryonic stem cells by DNA-mediated transfection, such as electroporation (Ausubel, et al., Current Protocols in Molecular Biology, John Wiley & Sons, 1999). Detailed procedures for culturing embryonic stem cells and methods of making transgenic mammals from embryonic stem cells may be found in “Teratocarcinomas and Embryonic Stem Cells, A practical Approach,” ed. E. J. Robertson (IRL Press, 1987).

In any of the foregoing methods of germline transformation, the construct may be introduced as a linear construct, as a circular plasmid, or as a viral vector which may be incorporated and inherited as a transgene integrated into the host genome. The transgene may also be constructed so as to permit it to be inherited as an extrachromosomal plasmid. The term “plasmid” generally refers to a DNA molecule that can replicate autonomously in a host cell.

The promoter is comprised of cis-acting DNA sequences capable of directing the transcription of a gene in the appropriate tissue environment and, in some cases, in response to physiological regulators. The promoter preferred for use in the present invention is derived from the αCaMKII gene, whose activity has been demonstrated to be restricted to the forebrain region (Mayford et al., Cell 81: 891-904, 1995). Other promoters are also known to direct the expression of exogenous genes to specific cell-types in the brain. Promoters useful for stem cell transformation, wherein tissue specificity is needed, include any promoter whose endogenous genes are expressed in the target cell of interest; e.g., the pkc7 promoter, the telencephalin promoter, the neuronal enolase promoter and the prp promoter. For somatic transformation, tissue specific promoters may or may not be needed. Thus, constitutive promoters, such as the CMV promoter or the β-actin promoter may be useful for somatic transformation.

Transgenic animals also may be obtained by infection of neurons either in vivo, ex vivo, or in vitro with a recombinant viral vector carrying an AC1 and/or AC8 gene. Suitable viral vectors include retroviral vectors, adenoviral vectors and Herpes simplex viral vectors, to name a few. The selection and use of such vectors is well known in the art.

The AC1 and/or AC8 transgenic animals of the invention may be used for in vivo assays. For instance, an in vivo assay, useful for identifying compounds that negatively affect expression of AC1 and/or AC8 in a mammal, comprises treating a transgenic animal that expresses an AC1 and/or AC8 transgene in its forebrain with a test compound suspected of down-regulating AC1 and/or AC8 function. The change in activity of the treated animal as compared with the untreated animal is then measured, a negative affect on the expression of AC1 and/or AC8 being indicative that the test compound down-regulates the expression of AC1 and/or AC8.

Suitable measurements include behavioral tests, such as of long term response to a persistent pain stimulus. This assay can be extended by measuring behavioral responses to persistent pain stimuli in the transgenic animals, inasmuch as such responses will be more robust in these animals as compared with non-transgenic animals, and differences caused by various test compounds will be more apparent.

It will be appreciated that assays similar to the in vivo assays discussed above can be developed easily in cultured cells. For instance, cultured neuronal or non-neuronal cells may be transformed with a DNA construct for expression of expression of AC1 and/or AC8 and those cells used for various biochemical and physiological assays to assess the changes resulting from the presence of the transgene. In another embodiment, cells or tissue slices from AC1 and/or AC8 transgenic animals may be utilized for a similar purpose.

The foregoing descriptions for methods of the present invention are illustrative and by no means exhaustive. The invention will not be described in greater detail by reference to the following non-limiting examples.

### EXAMPLES

The examples indicate that calcium-sensitive AC1 and AC8 are essential for central synapses to process sensory information in pathological conditions such as allodynia, just as their involvement in normal physiological conditions (such as memory). Furthermore, the examples provide evidence that cAMP-trigged CREB pathways are activated in pain-related brain areas including spinal dorsal horn, the ACC, and insular cortex, indicating that CREB-dependent plastic changes in synapses, as reported in other systems, also occur here.

A recent study in mice lacking CaMKIV, another kinase for triggering CREB activation in the nuclei, showed that CaMKIV is selectively required for fear memory, but not persistent pain. Since fear memory was significantly inhibited in AC1&8 DKO mice (Wong et al., 1999), the selectivity of CREB signaling pathways in physiological/pathological functions may depend on the upstream signaling molecules such as cAMP and CaMKIV.

Experimental Procedures

Mice. Adult, male mice lacking AC1, AC8 or AC1&8 and littermate wild-type (“WT”) mice were used (see Wong et al., 1999). Both WT and mutant mice were well groomed and showed no signs of abnormality or any obvious motor defects. No indication of tremor, seizure or ataxia was observed. It was impossible to distinguish mutant mice from wild-type mice and therefore all experiments were performed blind.

In situ hybridization. In situ hybridization experiments were performed as described in Wei, F., et al. (2001). AC1 and AC8 plasmids were digested with Hindl1l and reverse-transcribed using T7 RNA polyrneras (Promega). The brain and spinal slides were fixed in 4% paraformaldehyde and then treated with 50 μ/ml Proteinase K in PBS for 15 min. After washing twice in PBS, the slides were prehybridized in hybridization solution (50% Formamide, 5×SSC, 0.3 mg/ml Yeast tRNA, 100 μ/ml Heparin, 1×Denhardt's Solution, 10.1% Tween surfactants, 0.1% CHAPS, 5 mM EDTA, pH 8.0) for 4 hours at 60° C., followed by an incubation with 1 μ/ml of probe for a further 18 hours. After hybridization, slides were washed in 1×SSC at 60° C. for 10 minutes, 1.5×SSC at 60° C. for 10 minutes, then washed twice in 2×SSC at 37° C. for twenty minutes each, in 2×SSC containing 0.1 μ/ml RNAse A at 37° C. for 30 minutes, then in 2×SSC at room temperature for 10 minutes, in 0.2×SSC at 60° C. for 30 minutes twice, in 0.2×SSC at room temperature for 15 minutes, and once in PBT for 15 minutes. After washing, the slides were incubated in 20% heat-inactivated sheep serum in PBT for four hours at room temperature, and then incubated with pre-absorbed anti-digoxygenin antibody (Boehringer Mannheim) at 4° C. overnight. After antibody incubation, the slides were washed three times in PBT at room temperature for 30 minutes each and then in alkaline phosphatase buffer at room temperature for 5 minutes each. For every ml of alkaline phosphatase buffer (100 mM Tris, pH 9.5, 50 mM MgCl₂, 100 mM NaCl, 0.1% Tween 20), 1 μl of NBT (Nitro blue tetrazolium) and 3.5 gl of BCIP (5-bromo-4-chloro-3-indoyl phosphate) was added, and developed in the dark up to 18 hours. The slides were then washed twice in PBS to remove substrates and mounted with glycerol/PBS.

Adenylyl Cyclase Assay. Adenylyl cyclase activity in the spinal cord was assayed as described in Wong et al., 1999. Adenylyl cyclase activity levels are the means of triplicate determinations.

Behavioral experiments. Behavioral experiments were performed by observers (one for all acute tests and formalin test, another for the Complete Freund's adjuvant test (“CFT”)) who were blinded to the experimental situations of each animal. Behavioral allodynia was induced by CFA, 50% in saline, 10 μl; Sigma) injection into the dorsal surface of the left hind paw under halothane anesthesia as described in Wei et al., 2001. Mechanical sensitivity was assessed with a set of von Frey filaments (Stoelting; Wood Dale, Ill.). Based on preliminary experiments that characterized the threshold stimulus in untreated animals, the innocuous 0.4 mM (No. 2.44) filament, representing 50% of the threshold force, was used to detect mechanical allodynia. The filament was applied to the point of bending 6 times each to the dorsal surfaces of the left and right hindpaw. Positive responses included prolonged hindpaw withdrawal followed by licking or scratching. For each time point, the percent response frequency of hindpaw withdrawal was expressed as (number of positive responses)/6×100 per hindpaw. Hindpaw oedema was evaluated with a fine caliper at three days of CFA injection. Formalin (5%, 10 μl) was injected subcutaneously into the dorsal side of a hindpaw. The total time spent licking or biting the injected hindpaw was recorded during each 5 min interval over the course of 2 hr. The spinal tail-flick reflex was evoked by focused, radiant heat applied to underside of the tail. The latency to reflexive removal of the tail away from the heat was measured by a photocell timer to the nearest 0.1 sec. In the hot-plate test, mice were placed on a thermally-controlled metal plate (Columbia Instruments; Columbus, Ohio). The time between placement of a mouse on the plate and licking or lifting of a hindpaw was measured with a digital timer. Two different temperatures were used, 52.5 and 55.0° C. Mice were removed from the hot plate immediately after the first response. In all three tests, the mean response latency was calculated as the average of 3-4 measurements performed at 10 min intervals.

Immunocytochemistry. Mice were deeply anesthetized with 3-4% halothane and perfused through the ascending aorta with 50 ml of saline, followed by 200 ml of cold 0.1 M phosphate buffer (PB) containing 4% paraformaldehyde. Cryostat-cut brain sections (30 gm) were processed with anti-CaMKIV mouse antibody (1:500; Transduction Laboratories, Lexington, Ky.) and then with FITC-conjugated affinipure goat-anti-mouse IgG at 1:100 dilution (Jackson ImmunoResearch Laboratories, West Grove, Pa.). Images were obtained with Olympus Fluoview laser-scanning confocal microscope. Anatomical terminology is based on the atlas of Franklin and Paxinos (1997). The rostrocaudal levels of each sections corresponded to −1.70 to −2.18 mm (hippocampus), 0.98 to 0.5 mm (ACC), and 1.10 to 0.5 mm (insular cortex) relative to Bregma. Images were collected on Olympus BX60 microscopy and analyzed using NIH image (Scion Image). The integrated intensity for the selected appropriate regions was normalized to the corresponding integrated intensity in the adjacent white matter. Three measurements were made from three randomly selected non-contiguous sections of each nucleus per mouse observed from coded slides and averaged so that each animal had a mean value for regional pCREB immunoreactivity.

Data analysis. Results were expressed as mean±standard error of the mean (S.E.M.). Statistical comparisons were performed with the use of one- or two-way analysis of variance (ANOVA) with the post-hoc Scheffe F-test in immunocytochemical experiments, or the Student-Newmann-Keuls test in behavioral experiments, to identify significant differences. In all cases, P<0.05 was considered statistically significant.

### Example 1

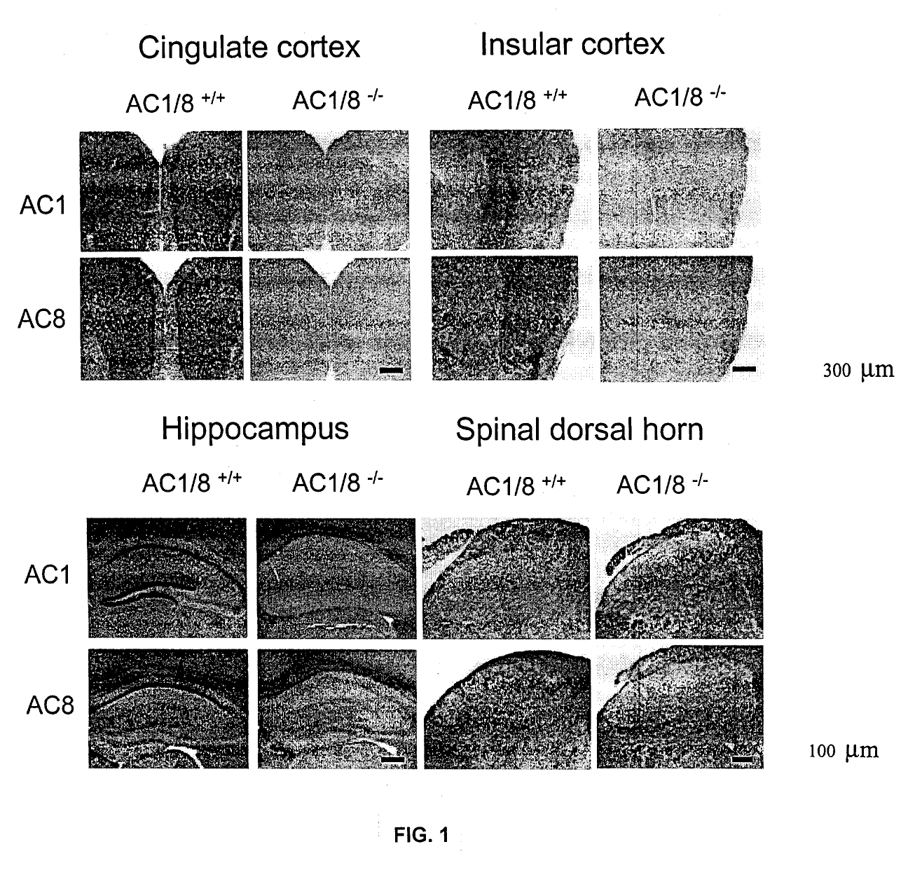

To examine the role of AC1 and AC8 in inflammation-related pain, the distribution of AC1 and AC8 in two pain-related forebrain areas, the anterior cingulate cortex (ACC) and insular cortex (Casey, 1999; Talbot et al., 1991; Rainville et al., 1997; Hutchinson et al., 1999) was studied. Both AC1 and AC8 were highly expressed in the ACC and the insular cortex of wild-type (WT) mice (n=2 mice for each group, FIG. 1). AC1 or AC8 were expressed throughout various layers of the ACC and insular cortex, no staining was seen in AC1&8 double knockout (DKO) mice. As shown in FIG. 1, AC1 and AC8 were also found in the hippocampus with different expression pattern. Additionally, in the spinal dorsal horn, a region critical for pain transmission and modulation (Perl, 1996; Fields et al., 1991; Urban and Gebhart, 1999), AC8 expressed at a moderate level and a low level of was detected (FIG. 1). In mice lacking AC1 or AC8 (n=2 mice), no significant changes in the expression level of the other calmodulin-regulated AC8 or AC1 (data not shown) were detected, indicating that the residual AC did not undergo compensation due to the removal of one AC. Therefore, it can be seen that AC1 and AC8 are both expressed in two pain-related forebrain areas, the anterior cingulate and insular cortex.

### Example 2

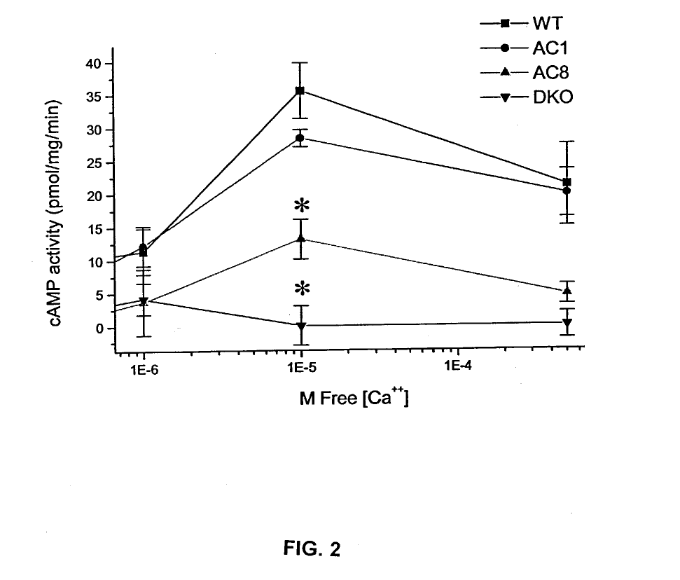

To determine the contribution of AC1 and AC8 to Ca²⁺-stimulated AC activity in the spinal cord, assays in the spinal cord of WT, AC1, AC8 and AC1&8 DKO mice (n=4 for each group) were carried out. While no significant changes were seen in AC1 KO mice, significant reduction in Ca^{z+}-stimulated AC activity was seen in AC8 KO mice. Furthermore, Ca²⁺-stimulated AC activity was completely blocked in AC1&8 DKO mice (FIG. 2). These results thus indicate that AC8 predominantly contribute to Ca²⁺-stimulated AC activity in the spinal cord. Therefore, AC1 and AC8 are both expressed at a low level in the spinal cord.

### Example 3

The roles of AC1 and AC8 in allodynia induced by hindpaw injection of CFA were tested. Application of a 0.4 mN von Frey fiber to the dorsum of a hindpaw elicited no response in untreated mice, but at one and three days after CFA injection (50%, 10 μl) into the dorsum of a single hindpaw, mice responded to stimulation of either the same (ipsilateral) or, to a lesser extent, the contralateral hindpaw by hindpaw withdrawal. This mechanical allodynia, or display of nociceptive response to a previously non-noxious mechanical stimulus, was significantly reduced in AC1 KO mice as compared with WT mice (n=5 for each group). No significant changes were seen in AC8 KO (n=5), indicating that ablation of AC8 alone, is not sufficient to affect allodynia. Because AC1 is expressed at a lower level in the spinal cord, these results suggest that AC1 in the ACC and insular cortex play roles in allodynia. Allodynia was completely abolished in AC1&8 DKO mice (n=5). Similar results were observed at the contralateral hindpaw. The hindpaw oedema was detected by measuring the hindpaw diameter and similar degree of inflammation were found in WT, AC1, AC8 and A1&8 DKO mice (n=5 for each group). Therefore, it appears that the deletion of Ca²⁺-CaM stimulated AC1 and AC8 completely block inflammation-related allodynia (non-noxious stimuli induce pain) in awaked mice.

The formalin pain test is a common test for responses to inflammation within a few hours (Dubuisson and Dennis, 1977; Haley et al., 1990). Typically, formalin induced phase 1 and phase 2 responses. In mice lacking AC1 (n=5) or AC8 (n=7), phase 2 was significantly decreased and greater reduction was observed in AC1&8 DKO mice (n=5, FIG. 2). Phase 1, was not affected in all three mutant mice. As previously reported (Wei et al., 2001), phase 3 was also recorded in WT mice (n=13). Phase 3 was not affected in AC1 or AC8 KO mice, but significantly reduced in AC1&8 DKO mice. To test if behavioral responses to acute noxious stimuli may be also affected, both the tail-flick reflex and hot-plate tests at 52.5 and 5.0° C. were performed. The responses in WT, AC1, AC8 and AC1&8 DKO mice were similar (the tail-flick test: WT, n=6; AC1 n=10, AC8, n=7, AC1&8, n=10; the hot-plate test: WT, n=10, AC1, n=6, AC8, n=7, AC1&8, n=6). Thus, acute pain is normal in AC1, AC8, and AC1&8 DKO mice.

Mice lacking AC1 and AC8 displayed no allodynia in chronic inflammatory pain model. This indicates that deletion of Ca²⁺-CaM stimulated AC1 and AC8 completely blocks inflammation-related allodynia (non-noxious stimuli induced pain). Furthermore, Phase 2 responses were dramatically reduced. In the formalin test, Phase 2 nociceptive responses were reduced to 10% of control mice in double knockout mice. In contrast, behavioral responses to thermal heating (noxious stimuli) were normal in single or double knockout mice, as well as Phase 1 responses in the formalin pain test were not affected, suggesting a selective role of Ca²⁺-sensitive AC1 and AC8 in the induction and expression of chronic pain.

Single deletion of AC1 or AC8 caused no or less changes in the same tests. Thus, it appears that AC1 and AC8 compensate each other in case of single genetic deletion. Due to their different sensitivities to Ca²⁺, their involvement in behavioral responses after tissue injury may vary. For example, in the allodynia test of the ipsilateral hind paw, AC1 deletion caused significant reduction, while AC8 deletion did not have a significant effect.

### Example 4

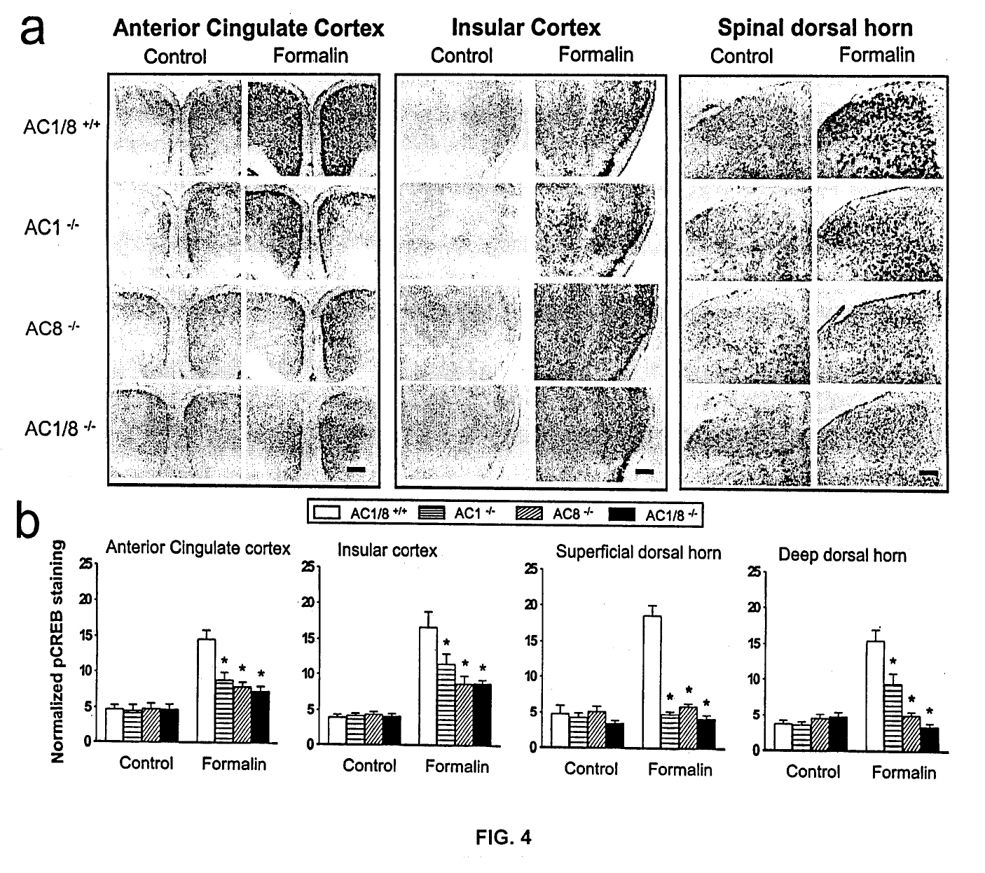

The cyclic AMP-responsive element binding protein (CREB) is a major transcription factor, which plays important roles in the formation of long-term memory in both invertebrates and vertebrates (see Silva et al., 1999; Tully, 1998). In the spinal cord and forebrains, CREB is activated after tissue injury or amputation (Ji and Rupp, 1997; Wei et al., 1999). Although the exact link between activation of CREB and persistent pain still unclear, pCREB can be used as a marker for activation of AC1 and AC8 during injury. In addition to cAMP, the calcium/calmodulin (CaM)-dependent protein kinase pathway also activates CREB (Bito et al., 1996; Sodering, 2000). To determine the contribution of Ca²⁺-stimulated CAMP to injury-activated CREB in the brain, if pCREB induced by hind paw formalin injection depends on AC1 and AC8 was tested. It was found that formalin injection activated CREB in spinal dorsal horn neurons of WT mice (n=8). In AC1, AC8 or AC1&8 DKO mice (n=5 for each group), pCREB in superficial dorsal horn neurons were completely abolished (FIG. 4). In deep dorsal horn neurons, pCREB was significantly reduced in AC1 KO mice (n=5) and more reduction in AC8 KO mice (n=5). As compared with mice receiving saline injection, pCREB was completely abolished in AC1&8 DKO mice. In support of cortical roles in pain, we found that CREB was activated in the ACC and insular cortex (FIG. 4). Interestingly, pCREB were reduced to similar extent in AC1, AC8 or AC1&8 DKO mice. There were significant residual pCREB even in AC1&8 DKO mice, indicating that Ca^{z+}-stimulated AC1 and AC8 are not the only pathways linked to CREB activation during injury. Therefore, it can be seen that activation of CREB by injury in pain-related central areas were significantly reduced or completely blocked.

Behavioral phenotypes are consistent with CREB activation in sensory related areas. Two major reasons contribute to this difference. First, cAMP may regulate synaptic functions without activating CREB, in particular for those, cAMP regulations that occur rapidly (for example, see, Rosenmund et al., 1994; Moss et al., 1992). Second, staining observed in the spinal dorsal horn (Example 1) may also have supraspinal modulatory components (see, Fields et al., 1991).

References

1. Xia, Z. and Strom, D. R. (1996) Calmodulin-regulated adenylyl cyclases and neuromodulation. Current Opinion in Neurobiology 7, 391-396.

2. Kandel, E. R. and Schawatz, J. H. (1982) Molecular biology of learning: modulation oftransmitter release. Science 218, 433-443.

3. Nestler, E. J. and Aghajanian, G. K. (1997) Molecular and cellular basis of addiction. Science 278, 58-63.

4. Malmberg, A. B., Brandon, E. P., Idzerda, R. L., Liu, H., McKnight, G. S. and Basbaum, A. I. (1997) Diminished inflammation and nociceptive pain with preservation of neuropathic pain in mice with a targeted mutation of the type I regulatory subunit of CAMP-dependent protein kinase. JJ Neuroscience. 17, 7462-7470.

5. Caterina, M. J. et al., (2000) Impaired nociception and pain sensation in mice lacking the capsaicin receptor. Science 288, 306-313.

6. Woolf, C. J. (1983) Evidence for a central component of post-injury pain hypersensitivity. Nature 306, 686-688.

7. Woolf, C. J. and Salter, M. W. (2000) Neuronal plasticity: increasing the gain in pain. Science 288, 1765-1768.

8. Cali, J. J., Parekh, R. S. and Krupinski, J. (1996) Splice variants of type VIII adenylyl cyclase. JBC 271, 1089-1095.

9. Casey, K. L. Forebrain mechanisms of nociception and pain: analysis through imaging. Proc. Mad. Acad. Sci. U.S.A. 96, 7668-7674 (1999).

10. Talbot, J. D., Marreft, S., Evans, A. C., Meyer, E., Bushnell, M. C. & Duncan, G. H. (1991). Multiple representations of pain in human cerebral cortex. Science 251, 1355-1358.

11. Rainville, P., Duncan, G. H., Price, D. D., Carrier, B. and Bushnell, M. C. (1997) Pain affect encoded in human anterior cingulate but not somatosensory cortex. Science 277, 968-971.

12. Hutchison, W. D., Davis, K. D., Lozano, A. M., Tasker, R. R. and Dostrovsky, J. O. (1999). Pain-related neurons in the human cingulate cortex. Nature Neuroscience 2, 403-405.

13. Wei, F. et al. Genetic enhancement of inflammatory pain by forebrain NR2B overexpression. Nature Neurosci. 4, 164-169 (2001).

14. Perl, E. R. in Neurobiology of Nociceptors (eds. Belmonte, C. & Cervero, F.) 5-36 (Oxford University Press, New York, 1996).

15. Field, 1991 Fields, H. L., Heinricher, M. M. and Mason, P., Neurotransmitters in nociceptive modulatory circuits. Ann. Rev. Neurosci. 14 (1991) 219-245.

16. Wong, S. T. et al. Calcium-stimulated adenylyl cyclase activity is critical for hippocampus-dependent long-term memory and late phase LTP. Neuron 23, 787-798 (1999).

17. Tully, T. (1998) toward a molecular biology of memory: the light's coming on! Nature Neuroscience 1, 543-545.

18. Silva, A. J., Kogan, J. H., Frankland, P. W. & Kida, S. CREB and Memory. Annu. Rev. Neurosci. 21, 127-148 (1998).

19. Ji, R. R. and Rupp, F. (1997) Phosphorylation of transcription factor CREB in rat spinal cord after formalin-induced hyperalgesia: relationship to c-fos induction. J Neurosci 17, 1776-1785.

20. Dubuisson, D. & Dennis, S. G. The formalin test: a quantitative study of the analgesic effects of morphine, meperidine, and brain stem stimulation in rats and cats. Pain 4, 161-174 (1977).

21. Haley, J. E., Sullivan, A. F. & Dickenson, A. H. Evidence for spinal N-methyl-D-aspartate receptor involvement in prolonged chemical nociception in the rat. Brain Res. 518, 218-226 (1990).

22. Wei, F., Li, P. & Zhuo, M. Loss of synaptic depression in mammalian anterior cingulate cortex after amputation. J. Neurosci. 19, 9346-9354 (1999).

23. Bito, H., Deisseroth, K., & Tsien, R. W. CREB phosphorylation and dephosphorylation: a Ca2+- and stimulus duration-dependent switch for hippocampal gene expression. Cell 87, 1203-1214 (1996).

24. Sodering, T. R. CaM-kinase: modulator of synaptic plasticity. Curr. Opin. Neurobiol. 10, 375-380 (2000).

25. Impey, S., Smith, D. M., Obrietan, K., Donahue, R., Wade, C & Storm, D. R. Nature Neurosci. 1, 595-601 (1998).

26. Moss, S. J., Smart, T. G., Blackstone, C. D. and Huganir, R. L. (1992) functional modulation of GABAA receptors by cAMP-dependent protein phosphorylation. Science 257:661-665.

27. Rosenmund, C., Carr, D. W., Bergeson, S. E., Nilaver, G., Scott, J. D. and Westbrook, G. L. (1994) Anchoring of protein kinase A is required for modulation of AMPA/kainate receptors on hippocampal neurons. Nature 368, 853-856.

28. Birren, S. J., Lo, L. C., and Anderson, D. J. (1993). Sympathetic neurons undergo a developmental switch in trophic dependence. Development 119, 597-610.

29. Franklin, K. B. J. & Paxinos, G. The Mouse Brain in Stereotaxic Coordinates. New York: Academic Press (1997).

We claim:

1. A method of down-regulating an activity associated with AC1, AC8 or both in a subject comprising administering to the subject an antagonist to AC1, AC8 or both in an amount sufficient to affect the antagonism.

2. The method in accordance with claim 1 wherein the activity associated with AC1, AC8 or both is an activity in the forebrain.

3. The method in accordance with claim 2 wherein the activity associated with AC1, AC8 or both is persistent pain.

4. The method in accordance with claim 3 wherein the persistent pain is inflammation related allodynia.

5. A method of inhibiting persistent pain in a patient in need of such treatment comprising administering to the patient a therapeutically effective dose of an antagonist to AC1, AC8 or both in an amount sufficient to inhibit the persistent pain in the patient.

6. The method in accordance with claim 5 wherein the persistent pain is inflammation related allodynia.

7. A method of down-regulating an activity associated with AC1, AC8 or both in a subject comprising reducing or eliminating expression of AC1, AC8 or both at the transcription level the translational level or both levels.

8. The method in accordance with claim 7 wherein the activity is down-regulated using a promoter derived from the αCaMKII gene.

9. A method of down-regulating an activity associated with AC1, AC8 or both in a subject comprising the selective use of a compound that acts inside the subject's cells to interfere with the interaction between AC1, AC8 or both and their down stream targets.

10. The method in accordance with claim wherein the cells are cells of the subject's forebrain.

11. The method in accordance with claim 10 wherein the compound is comprised of somatic cells transformed with a vector encoding an antisense molecule or ribosome, or a transcription suppressing protein designed to inhibit expression of AC1, AC8 or both.

12. A method of identifying compounds that inhibit persistent pain by down-regulating AC1, AC8 or both activity comprising
contacting a chimeric DNA construct comprising an AC1, AC8 or both promoter operably linked to a reporter gene with a test compound suspected of down-regulating AC1, AC8 or both and then
measuring expression of the reporter gene, a decrease in the expression of the reporter gene in the presence of the compound being indicative that the compound inhibits persistent pain.

13. A genetically altered non-human animal having increased sensitivity to persistent pain as compared with an equivalent, but unaltered animal, wherein the animal expresses a gene encoding AC1, AC8 or both to a greater extent in the forebrain than does the equivalent, but unaltered animal.

14. The genetically altered animal in accordance with claim 13 wherein the genetically altered animal overexposes an endogenous gene encoding AC1, AC8 or both.

15. The genetically altered animal in accordance with claim 13 wherein the genetically altered animal expresses a transgene encoding AC1, AC8 or both.

16. The genetically altered animal in accordance with claim 15 wherein the transgene includes the entire coding region of an AC1, AC8 or both gene, or its complementary DNA or chimeric genes containing part or all of an AC1 and/or AC8 coding region.

17. The genetically altered animal in accordance with claim 16 wherein the gene is a transgene includes a promoter derived from the αCaMKII gene.

18. An in vivo assay for identifying compounds that inhibit persistent pain by down-regulating AC1, AC8 or both activity comprising:
administering to a non-human transgenic animal that expresses a gene encoding AC1, AC8 or both to a greater extent in its forebrain than does the equivalent, but unaltered animal a test compound suspected of down-regulating AC1, AC8 or both and then
directly or indirectly measuring an activity associated with AC1, AC8 or both of the treated animal as compared with an equivalent untreated animal, a decrease in the activity of the treated animal being indicative that the test compound reduces or eliminates persistent pain.

19. The method in accordance with claim 18 wherein activity is measured using behavioral tests of long term response to a persistent pain stimulus.
